(19) United States
(12) Patent Application Publication
(10) Pub. No.: US 2011/0052652 A1
(43) Pub. Date: Mar. 3, 2011

(54) Solid pharmaceutical composition comprising agglomerated nanoparticles and a process for producing the same

(76) Inventors: Henry Jun Suzuki, Vila Leopoldina (BR); Dante Alario Júnior, Chacara Santo Antonio (BR)

(21) Appl. No.: 12/093,410

(22) Filed: May 12, 2008

Related U.S. Application Data
(63) Continuation of application No. PCT/BR2006/000247, filed on Nov. 13, 2006.

(30) Foreign Application Priority Data
Nov. 11, 2005 (BR) PI 0505479-6

(51) Int. Cl. A61K 9/14; A61K 9/51; A61K 9/20; A61K 9/00; A61P 31/00; B29B 9/08; B29C 35/16

(52) U.S. Cl. 424/405; 424/489; 424/451; 424/499; 424/501; 424/464; 264/6; 264/28

(57) ABSTRACT

The present invention relates to pharmaceutical compositions comprising at least one active ingredient delivered by a nanoparticle. More specifically, the invention relates to solid pharmaceutical compositions comprising nanoparticles, wherein the nanoparticles are in the form of agglomerates with elevated equivalent aerodynamic diameters. The invention further relates to a process for producing such nanoparticles.

### FIELD OF THE INVENTION

The present invention relates to a pharmaceutical composition. More specifically, to a solid pharmaceutical composition comprising nanoparticles, wherein the nanoparticles are in the form of agglomerates with elevated equivalent aerodynamic diameter, as well as, to a process for producing the same.

### BACKGROUND OF THE INVENTION

The recent development of technologies for production and application of nanoparticles bearing pharmaceutically activated molecules showed a broad of alternative choices for the formulation of new drugs.

Among several types of nanoparticles employed in pharmaceutical compositions, we must highlight the polymeric nanoparticles. Polymeric nanoparticles are drug carrier systems with a mean diameter lower than 1 micrometer, in which the active ingredient is kept, in encapsulated or adsorbed form. The term nanoparticles can be used with the meaning of nanospheres and nanocapsules.

Nanospheres are made by a polymeric matrix in which the active ingredient is kept or adsorbed. In the mean time nanocapsules are constituted by a polymeric shell built around a core, the active ingredient being able to be contained inside the core or over the covering shell.

Generally, the process for producing of polymeric nanoparticles can be classified as in-situ polymerization methods or using of pre-formed polymer methods. Materials usually employed for nanoparticles preparation are, for example: polymers of alkyl cyanoacrylates, copolymers of (meth)acrylic acid and acrylic or (meth)acrylic esters (Eudragits), polymers and copolymers of lactic acid and glycolic acid (TIP and PLGA) and poly(ε-caprolactone) (PCL).

Industrial application of polymeric nanoparticles compositions in pharmaceutical formulations has the nanoparticles instability in liquid medium as one of its technical barriers as a function of problems, such as nanoparticles aggregation, polymeric materials or active ingredients decomposition, the changing of nanoparticles physical-chemical properties along the time or even the incompatibility of nanoparticles with excipients usually employed in pharmaceutical compositions; specially, in liquid or semi-solid formulations.

Having in mind the fact that nanoparticles stability problems may be minimized by a compositions drying step, the development of pharmaceutical solid forms has shown itself as an alternative for polymeric nanoparticles-based commercial formulations possibilities. Process normally employed for obtaining of polymeric nanoparticles solid compositions envolves drying methods such as concentration by evaporation, spray-drying or freezing-drying.

In this context, deserves distinction the existence of several issues reporting the obtention process of nanoparticles solid compositions, wherein distinction is given to the obtention of particles with reduced dimensions.

WO 9625152 A1 (Nanosystems LLC) discloses the process for obtaining of solid nanoparticles with particles size averages under 400 nanometers, by utilization of microfluidizer. EP275796 A1 (National de La Recherche Cientifique Centers) discloses the process for obtention of solid nanoparticles with particles size average under 500 nanometers, by liquid phase preciptation. U.S. Pat. No. 5,573,783 (Nanosystems Inc) discloses coated nanoparticles with diameter between 150 and 250 nanometers. EP 601619 A2 discloses the use of surface modification agents acting as stabilizers for nanoparticles formulations, avoiding its agglomerating during sterilization process. US 2002/068092 (Elan Pharma International Ltd.) also discloses the use of cationic surface modification agents for prevention of nanoparticles aggregation. Processes for production of average aerodynamic diameter size from about 2 to 3 micrometers nanoparticles agglomerates or collecting, are disclosed, for example, by Pandey R et al., (“Poly (DL-lactide-co-glycolide) nanoparticle based inhalable sustained drug delivery system for experimental tuberculosis”. J. Antimicrob. Chemother.; December 2003; 52 (6): 981-6) and Sham J. O. et al., (“Formulation and characterization of spray-dried powders containing nanoparticles for aerosol delivery to the lung”. Int. J. Pharm., Jan. 28, 2004; 269(2): 457-67).

Adversely if drying nanoparticles compositions resolves in great part stability problems, on the other hand it has the inconvenience of increasing personal and environmental exposition risk to nanoparticulated materials.

As a function of usually desirable physic-chemical properties and reduced particle size in nanopoarticles composition (for example, surface repulsion avoiding agglomerate formation), formulations of nanoparticles in dry powder form not only can be easily suspended and kept in suspension in environment, but also can penetrate deeply in airways; increasing, consequently, the risk of pulmonary and systemic exposition both for final formulation users and for professionals envolved on its production and handling.

Based on alternative studies to solve personal and environmental exposition problems during production, handling and application of solid compositions based on nanoparticles, the present inventors disclosed that it is possible to reduce risks of exhibition to nanoparticles when these are delivered in the form of agglomerates with large dimensions.

### DESCRIPTION OF THE INVENTION

In a first aspect, the present invention relates to a solid pharmaceutical composition comprising nanoparticles, wherein the nanoparticles are delivered essentially in the form of agglomerates with large dimensions. More specifically, to a pharmaceutical composition comprising at least one active ingredient delivered in nanoparticles, wherein more than 90% of the amount of active ingredient is kept in nanoparticles agglomerates with aerodynamic equivalent diameter higher than or equal to 2.5 micrometers (DA₉₀%≧2.5 micrometers); preferably, the invention relates to a composition comprising at least one active ingredient delivered in nanoparticles, wherein more than 99% of the amount of active ingredient is kept in nanoparticles agglomerates with aerodynamic equivalent diameter higher or equal to 10 micrometers (DA₉₉%≧10 micrometers).

A solid composition comprised by the present invention is disclosed, for example, in “Remington: The Science & Practice of Pharmacy” (2000) 21. ed., Mack Publishing Company; such as: powders, granulated, microgranules, microspheres, capsules, pills, paevenes and tablets. The composition of the present invention can be both in final form and intermediary form for preparation of other compositions (for example, powder for pills manufacture). Powder or granulated compositions with particle size lower than 1 millimeter, specially powders or granulated ready for topical dermatological, transmucosal, or even, for the treatment of open wounds application, correspond to interesting application forms for use in the present invention.

According to the present invention, the term “nanoparticles” correspond to carrier systems for drugs, in which at least one active ingredient is kept, encapsulated or adsorbed, and that exhibit a diameter lower than 1 micrometer. Preferably, used nanoparticles are polymeric nanoparticles, in the form of nanospheres or nanocapsules. Nanoparticles of any nature is comprised by the present invention; specially interesting is the use of polymeric solid nanoparticles comprising surface modification agents that promote nanoparticles dispersion after its application at administration site or the mixture of the compositions with dilution liquid agents. Examples of surface modification agents are described, for example, in WO 9126635 A2 (Bosch W H et al.; Elan Pharma International Ltd.).

The term “equivalent aerodynamic diameter” corresponds to the diameter of a spherical hypothetical particle of unitary density (1 g/m3) which has the same final sedimentation speed of the particle in the air regardless of its actual geometrical size, form or density.

According to the present invention, the term “agglomerate” is applied to nanoparticles physically binded sets, preferably, by the use of a material bridge formed by substances that unite nanoparticles. Alternatively, nanoparticles agglomerates can be formed, for example, as a result of nanoparticles electrostatic self attraction or by the use of a physical support over which the nanoparticles are deposited.

In accordance with a preferably aspect, the nanoparticles agglomerates are formulated in such a way that they keep large dimensions during production and handling processes and enter into disaggregation when in contact with the application site (for example, skin, membranes) or after the mixture with liquid or semi-solid vehicles. Therefore, it is especially interesting the use of water soluble materials as binding agents for agglomerate formation.

Examples of substances that can be used to agglomerate nanoparticles are: materials with zeta electrostatic potential or zeta reverse potential to that of nanoparticles, polymeric and non-polymeric adhesive materials (as ion exchange resine, cellulose polymers, cellulose polymer ethers and cellulose polymer hydroxyalkylethers, polyethyleneglycol, polyvinylpyrrolidone, polymers and copolymers of (meth)acrylic acid, sugars, organic and inorganic salts).

Agglomerates also may comprise a physical support over which nanoparticles are deposited. Examples of such physical supports are: silicon dioxide, talcum powder, starch, zinc oxide, titanium dioxide; which can be directly contacted with nanoparticles or, optionally, be previously covered by an intermediary layer.

The active compound amount determination inside particles with aerodynamic equivalent diameter lower than 2.5 (PM_{2.5}) or 10 (PM₁₀) micrometers can be done in the direct form, on basis of active compounds dosage in particles with such range of aerodynamic diameter or, non-directly, on basis of the difference between total amount of active compounds and active compounds in particles with equivalent aerodynamic diameter higher than 2.5 (PM_{2.5}) or 10 (PM₁₀) micrometers. Separation of particles with different diameters can be done by using membranes or calibrated filters for suspension particles used in equipments for the determination of particulated materials PM₁₀ and PM_{2.5}.

In a second aspect, the present invention refers to a process for the production of a pharmaceutical composition comprising nanoparticles agglomerates, which comprising a step for nanoparticles formation in suspension followed by a step of nanoparticles suspension drying and agglomerates formation and comprising even, at least a step for measuring the aerodynamic equivalent diameter of dry suspension resulting particles (including free or agglomerated ones) for checking whether at least 90% of whole nanoparticles agglomerates are with aerodynamic equivalent diameter higher or equal to 2.5 micrometers; preferably, at least 99% of such nanoparticles agglomerates are with aerodynamic equivalent diameter higher or equal to 10 micrometers.

According to the present invention, the nanoparticles formation step is non-limited to specific processes. Examples of such processes that can be employed for such nanoparticles formation are: emulsion/evaporation, double emulsion/evaporation, salting-out, emulsifying-diffusion, solvent striping/nanoprecipitation and emulsion/diffusion/evaporation; as described, for example, in Bullet I. et al., (Critical Reviews in Therapeutic Drug Carrier Systems, (2004) 21 (5): 387-422).

According to the present invention, the nanoparticles drying step for agglomerates formation can be achieved through several processes, with no limitation. Examples of the above-mentioned step are the simple evaporation, freeze-drying or spray drying of the suspensions containing the nanoparticles. Preferably, the process is the spray-drying process, using a physical support and water soluble substances for nanoparticles collection or aggregation. Examples of such processes for nanoparticles agglomerates production are described, for example, in WO 0027363 (Bosch HW; Nanosytem).

According to the present invention, the step of particles equivalent aerodynamic diameter measurement is carried out to check whether at least 90% of whole nanoparticles agglomerates are with aerodynamic equivalent diameter higher or equal to 2.5 micrometers; preferably, at least 99% of such nanoparticles agglomerates are with aerodynamic equivalent diameter higher or equal to 10 micrometers.

In accordance with a preferably aspect for carrying out the present invention, the step for measurement of aerodynamic equivalent diameter of resulting particles from nanoparticles suspension drying step can accomplished by using equipments as, for example, “Mastersizer S” and “Masterseizer 2000” (Malvern), coupled to dry powder feeder; preferably, dry powder feeder provided with particle dispersors able to disaggregate agglomerates with relatively low mechanical resistance, for example, as “MS-64; dry Powder Feeder unit—QS” (Malvern).

Preferably, nanoparticles suspensions drying process comprised by the present invention must essentially produce nanoparticles agglomerates free of agglomerates with dimensions lower then 2.5 or 10 micrometer. According, when the step for measurement of nanoparticles aerodynamic equivalent diameter show the existence of small particles lower than the specified limits, the product will be disapproved; being alternatively re-processed till achieve the expected size particle specifications.

The present invention has no limitations regarding chemical or pharmacological nature of active ingredients to be delivered by nanoparticles. Therefore, the compositions and processes comprised by the present invention are specially addressed for transportation of drugs that could show pulmonary or systemic exhibition risks, such as antibiotics, citostatic agents or immunosuppression agents. In the case of dermatological topical use, the compositions according the present invention may be especially useful for conveying antifungal, antibiotic or antiseptic agents, for external use, in the form of powders or talcum powders ready for use.

The following are described experimental examples for illustrate the present invention without, therefore, limiting its scope:

### Example 1

### Process for Dry Powder Production with Aerodynamic Equivalent Diameter DA₉₉%≧10 Micrometers, Containing Nanoparticles Cluster, Comprising Two Steps for Aerodynamic Equivalent Diameter Measurement

An aqueous nanoparticle PLGA suspension with average diameter of about 300 nanometer containing a drug A, is freezing-dryed, with the use of 2.5 parts of nanoparticles amount-based manitol, as crioprotecting agent. 30 grams of freezing-dryed product are subjected to aerodynamic apparent diameter measurement with the use of a Malvern Masterseizer S equipment, coupled to an air jet dry powder dispersor “MS-64; Dry powder feeder unit—QS” (Malvern) calibrated for an atomization pressure of 2 bar. The results obtained for measurement of aerodynamic diameter indicates that more than 1% of the whole sample is in the form of particles with aerodynamic equivalent diameter lower than 10 micrometers, the freezing-dryed product is disapproved. The freezing-dryed disapproved product is then resuspended in water (20 parts of water) and, then, is added to a suspension of 0.5 parts of colloidal silicon dioxide based on whole freezing-dryed product. The obtained suspension is then subjected to a spray-drying process for the production of a dry powder. 30 grams of the spray-dryed product by are subjected again to an aerodynamic equivalent diameter measurement with the use of a Malvern Masterseizer S equipment, according to above. The result of aerodynamic diameter obtained measurement indicates that more than 99% of the whole sample is in the form of particles with aerodynamic equivalent diameter higher than 10 micrometer, product is then approved.

### Example 2

### Process for Production of Dry Powder with Aerodynamic Equivalent Diameter DA₉₉%≧10 Micrometers, Containing Nanoparticle Cluster, Comprising One Step of Aerodynamic Equivalent Diameter Measurement

Dry powder is produced by spray-drying, according to the example 1, except by the fact that the freeze-drying and measurement of particle size steps are moved out. 30 grams of spray-dryed product are subjected to a aerodynamic equivalent diameter measurement step with the use of a Malvern Masterseizer S equipment, coupled to an air jet dry powder dispersor “MS-64; Dry powder feeder unit—QS” (Malvern) calibrated for an atomization pressure of 2 bar. The result of aerodynamic diameter obtained measurement indicates that more than 99% of the whole sample is in the form of particles with aerodynamic equivalent diameter higher than 10 micrometer, the product is finally approved.

All the publications above mentioned in this descriptive report are here incorporated by reference. Several modifications and variations of the present invention are evident for those skilled in the art, without departing from the scope and the spirit of the invention.

1-11. (canceled)

12. A solid pharmaceutical composition comprising at least one active ingredient delivered in nanoparticles, wherein said nanoparticles are in the form of agglomerates with high equivalent aerodynamic diameter.

13. The composition of claim 12, wherein more than 90% of the amount of active ingredients delivered in nanoparticles is kept in nanoparticles agglomerates with equivalent aerodynamic diameter higher than or equal to 2.5 micrometers (DA90%≧2.5 micrometers).

14. The composition of claim 12, wherein more than 99% of the amount of active ingredients delivered in nanoparticles is kept in nanoparticles agglomerates with equivalent aerodynamic diameter higher than or equal to 10 micrometers (DA99%≧10 micrometers).

15. The composition according to claim 12, wherein the nanoparticles are nanospheres or nanocapsules.

16. The composition of claim 15, wherein said nanoparticles are polymeric.

17. The composition of claim 12 wherein the nanoparticle agglomerates are formed using binding agents.

18. The composition of claim 17 wherein said binding agents are selected from the group consisting of: materials with zeta electrostatic potential or zeta reverse potential to that of nanoparticles, and at least one polymeric or non-polymeric adhesive material.

19. The composition of claim 18 wherein said agglutinant agent is water soluble.

20. The composition of claim 18 wherein the adhesive material is selected from the group consisting of: ion exchange resin, cellulose polymers, cellulose polymer ethers and cellulose polymer hydroxyalkylethers, polyethyleneglycol, polyvinylpyrrolidone, polymers and copolymers of (meth)acrylic acid, sugars, organic and inorganic salts.

21. The composition of claim 20 wherein said adhesive material comprises mannitol.

22. The composition of claim 18 wherein the nanoparticle agglomerates are formed by using at least one physical support over which nanoparticles are deposited.

23. The composition of claim 22 wherein the physical support is selected from a group consisting of: silicon dioxide, talcum powder, starch, zinc oxide, titanium dioxide.

24. The composition of claim 12 wherein said composition is in the form of powders, granulated, microgranules, microspheres, capsules, pills, paevenes and tablets.

25. The composition of claim 24 wherein said composition is in the form of powder or granulated ready for topical dermatological, transmucosal application or for the treatment of open wounds.

26. The composition of claim 24 wherein said powder is used as an intermediate for the preparation of other pharmaceutical compositions.

27. A composition according to any of claims 12 wherein the equivalent aerodynamic diameter of nanoparticle agglomerates is smaller than 1 millimeter.

28. The composition of claim 12 wherein the active ingredient comprises at least one antifungal, antibiotic or antiseptic agent delivered in nanoparticles.

29. A process for the production of a pharmaceutical composition comprising nanoparticle agglomerates, wherein said method comprises: (a) a step of producing nanoparticles in suspension; (b) at least one step of drying the nanoparticles suspension and forming the agglomerates; and (c) at least one step of measuring the equivalent aerodynamic diameter of the nanoparticles agglomerates obtained from the drying of the suspension.

30. Process according to claim 29, wherein the step of aerodynamic diameter measurement is performed to assure that at least 90% of the amount of such particles has an equivalent aerodynamic diameter higher or equal to 2.5 micrometers.

31. Process according to claim 29, wherein the step of aerodynamic diameter measurement is performed to assure that at least 99% of the amount of such particles has equivalent aerodynamic diameter higher or equal to 10 micrometers.

32. Process according to claim 29 further comprising at least one step for resuspending the agglomerates followed by the repetition of steps: (b) drying the nanoparticles suspension and forming of agglomerates; and (c) measuring the equivalent aerodynamic diameter of nanoparticles agglomerates obtained from the drying of the suspension until at least 90% of the amount of such particles has an equivalent aerodynamic diameter higher or equal to 2.5 micrometers.

33. Process according to claim 29 further comprising at least one step for resuspending the agglomerates followed by the repetition of steps: (b) drying the nanoparticles suspension and forming of agglomerates; and (c) measuring the equivalent aerodynamic diameter of nanoparticles agglomerates obtained from the drying of the suspension until at least 99% of the amount of such particles has equivalent aerodynamic diameter higher or equal to 10 micrometers.

34. Process according to claim 29 wherein the measurement step is performed with the nanoparticles agglomerates in its dry-powder form.

35. Process according to claim 29 wherein the drying and agglomerate forming step is performed by: simple evaporation, freeze-drying or spray drying of the suspensions containing the nanoparticles.

36. Process according to claim 35 wherein the drying and agglomerate forming step is preferably performed by spray drying of the suspensions containing the nanoparticles.

37. Process according to claim 29 wherein the step of measuring the equivalent aerodynamic diameter of the particles obtained from the drying of the nanoparticles is performed using specific equipments coupled to dry powder feeders; preferably, dry powder feeders provided with particle dispersors.
